(19) United States
(12) Patent Application Publication
(10) Pub. No.: US 2002/0026230 A1
(43) Pub. Date: Feb. 28, 2002

(54) Removable stent

(75) Inventors: Franciscus Laurens Moll, La Bosch En Duin (NL); Menno Kalmann, Elspeet (NL)

Correspondence Address: FINNEGAN, HENDERSON, FARABOW,, GARRETT & DUNNER, L.L.P., 1300 I STREET, N.W., WASHINGTON, DC, 20005-3315, US

(73) Assignee: ATROPOS Limited

(21) Appl. No.: 09/910,008

(22) Filed: Jul. 23, 2001

Related U.S. Application Data
(63) Continuation-in-part of application No. PCT/NL99/00779, filed on Dec. 18, 1999.

(30) Foreign Application Priority Data
Feb. 5, 1999 (NL) 1011232
Apr. 27, 1999 (NL) 1011903
Feb. 13, 2001 (IE) 2001/0114
Jul. 21, 2000 (IE) 2000/0582

(51) Int. Cl. A61F 2/06

(52) U.S. Cl. 623/1.13; 623/1.17

(57) ABSTRACT

Device suitable for internally supporting vessels in particular circular vessels in the non-medical and medical fields, such as transmission pipes and blood vessels, the urinary tract, the digestive tract, and airways, said device comprising;

an outer wall, and

an inner wall in association with the outer wall, whereby both the inner and outer wall are expandable and contractible between an expanded support position, in which support position the outer wall contacts an internal surface of the vessel to be supported and a contracted displacing position wherein the device is displaceable to and from a pre-desired location in the vessel, and,

releasable locking means for releasibly locking the device in the expanded support position and/or the contracted displacing position.

### INTRODUCTION

The present invention relates to a device suitable for internally supporting vessels, in particular circular vessels in the medical and non-medical fields, an assembly comprising said device, means for introducing and/or removing said device into/from the vessel, to a process for arranging the device within a vessel and to the use of said device and assembly.

The collapse of vessels, such as transmission pipes, for example water and fuel pipes, is very problematical and in some circumstances can lead to dangerous consequences.

A serious medical problem is the silting up of blood vessels, for instance with calcium, this being called arteriosclerosis. This can lead to a blockage of the blood vessel, called stenosis.

Stenosis of blood vessels can cause a complete blockage of the blood vessel which leads to serious health consequences, for example circulatory problems, for the sufferer, whereby a rapid deterioration in health ensues. Advanced stenosis if not operated upon can cause wastage and death of body tissue necessitating in certain cases, in amputation.

Inflatable, tubular prostheses are known, which can be inserted into blocked tubular organs and subsequently expanded in order to re-open these organs.

Further since such tubular prostheses, commonly referred to as stents, are made from material alien to the body, it is often necessary to remove the stent once the acute situation has been treated. Otherwise there exists a very real danger of thromboses and infections resulting from bodily rejection of the stent material.

Such prostheses, or stents, are currently surgically removed, often during a complicated operation carried out under narcosis. If this however presents difficulties or possible dangerous consequences to the patient, the stents are allowed to remain within the patient lead to the above consequences.

An object of the present invention is to overcome one or more of the above mentioned non-medical and/or medical problems.

According to a first aspect invention, there is provided a device according to any of the claims 1-17.

The device according to the present invention can therefore be arranged between an expanded, locked, arrangement wherein the vessel is itself expanded and held open, and the device can also be released from this expanded, locked, arrangement into a contracted arrangement wherein the device may be displaced into the vessel or removed therefrom simply by guiding it through the vessel concerned.

The stent can also be provided with, for example, impregnated with a medicament, to act as a drug delivery system, whereby the drug can be very accurately dosed directly at the area to be treated, before being removed.

Furthermore, the stent can be re-useable.

The stent may be provided with a medical tracer and/or radioactively ‘loaded’ in order to provide accurate medical diagnosis, i.e. by means of imaging, and/or very accurate, localized radiation therapy.

The stent can be covered with a thin sheet/craft, i.e. a thin prosthesis for a blood vessel, urinary tract or such like. This sheet/craft can be elastically arranged around the stent. On expansion of the stent, the craft also expands whereby the stent craft is displaced. On shrinkage of the stent, the sheet/craft also shrinks and can therefore be removed along with the stent. This can therefore be considered as a removable craft or a removable stent craft.

A non-elastic sheet/craft can also be arranged around the stent.

When the stent is then expanded, the sheet craft is again pushed against the wall of the lumen. However, on removal of the stent, this sheet craft remains behind.

This can be very important, for example in the following application:

A prosthetic for the inner wall of a blood vessel can be arranged thus. After a few days, the prosthetic has grown onto the blood vessel inner wall, whereby the stent is now superfluous. The stent is removed and the sheet craft remains behind.

According to a second aspect of the present invention there is provided an assembly according to claims 18 or 19.

By means of such an expandable/deflateable balloon catheter for example, the device can be easily arranged between its expanded locked arrangement and its contracted, displacing arrangement.

According to a third aspect of the current invention there is provided a process according to claims 20 or 21.

Such a process for introducing the above device, arranging the device in its expanded treating arrangement, and once the treatment period has expired, re-arranging the device in its contracted displacing position, whereby the device can be brought into and removed from the vessel along the same route provides an extremely effective, easy method for treating vessels, whereby the stent can be safely, efficiently and quickly inserted into/removed from a body vessel, for example.

According to further aspects of the present invention there is provided a method according to claim 22, and the uses according to claims 23-28.

According to one aspect there is provided a method for preparing an expandable stent comprising the steps of:

forming a generally spiral shaped element having a first free end and a second free end, the spiral element having a contracted configuration in which the first free end is an inner free end and the second free end is an outer free end; and

reversing the spiral so that the first free end becomes an outer free end and the stent is pretensioned.

In another aspect the invention provides a method for operating an expandable stent in the form of a generally spiral-shaped element the method comprising the steps of:

delivering the stent in the pretensioned configuration to a desired site;

deploying the stent by expanding the stent to a first expanded configuration; and

retrieving the stent by expanding the stent further to a second expanded configuration.

In a further aspect the invention provides a method for manipulating a stent having a pretensioned contracted configuration, a first expanded configuration and a second expanded configuration, the method comprising the steps of:

delivering the stent in a pretensioned contracted form to a desired site;

deploying the stent by expanding the stent to the first expanded configuration; and

subsequently retrieving the stent by further expanding the stent to the second expanded configuration whereby the stent collapses.

In one embodiment of the invention the pretensioned contracted stent is mounted on an introduction balloon catheter and the method includes the steps of advancing the balloon catheter to a desired site and inflating the introduction balloon to deploy the stent. Preferably the stent is retrieved by advancing a retrieval balloon catheter to the stent and inflating the retrieval balloon to expand the deployed stent to the second expanded configuration whereby the stent collapses.

According to another aspect there is provided an expandable stent comprising a generally spiral shaped element having a pre-tensioned configuration for delivery to a desired site, a first expanded configuration for deployment of the stent, and a second expanded configuration for retrieval of the stent, the stent having a medicament delivery system for delivery of a medicament at a target site.

In one embodiment the medicament system comprises a coating containing a medicament.

Preferably the medicament delivery system comprises a craft or tissue containing a medicament. The tissue or craft may be of a layered construction.

In one embodiment the tissue or craft comprises a first layer for drug delivery in one direction and a second layer to prevent drug delivery in an opposite direction.

In one arrangement the stent comprises a number of rings extending from a spine and the craft or tissue extends at least some of the adjacent rings.

In a further aspect the invention provides a method for delivery of a drug medicament to a target site comprising

providing a stent with a medicament delivery system;

delivering the stent to a desired site;

deploying the stent to deliver the medicament; and

retrieving the stent.

The invention will now be further clarified by way of the following description which refer to the figures wherein:

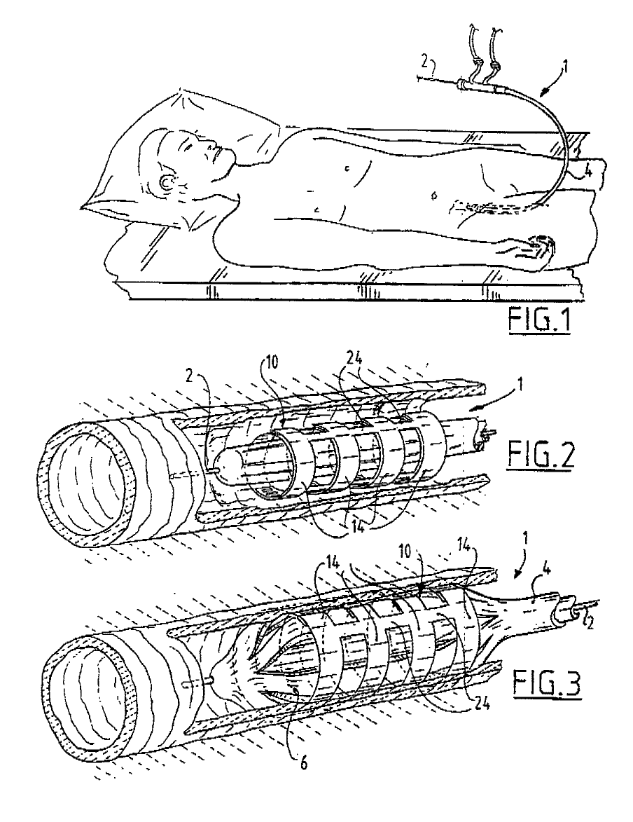

FIG. 1 shows a partially cut away perspective view of the arranging/removal of the device according to the present invention;

FIG. 2 shows a perspective partially cut away view of the device within a blood vessel in its contracted arrangement around a deflated balloon catheter;

FIG. 3 shows the arrangement in FIG. 2 whereby the device occupies its expanded, treating arrangement;

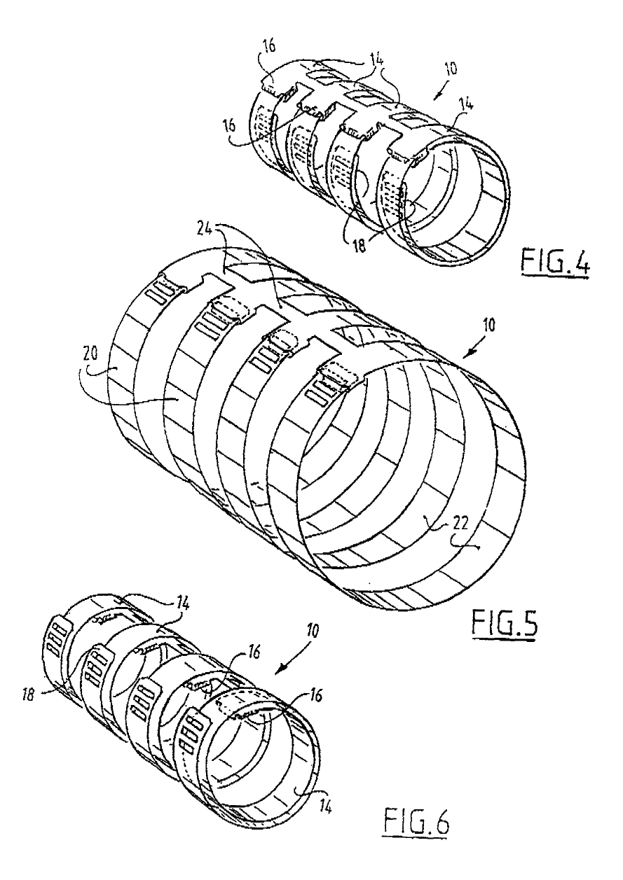

FIG. 4 shows a perspective view of this first preferred embodiment of the device in a first contracted arrangement, in the position when arranged for insertion on a balloon catheter;

FIG. 5 shows the embodiment in FIG. 4 occupying its expanded, locked treating arrangement;

FIG. 6 shows a further perspective view of the device in a second contracted arrangement when arranged for removal on a balloon catheter;

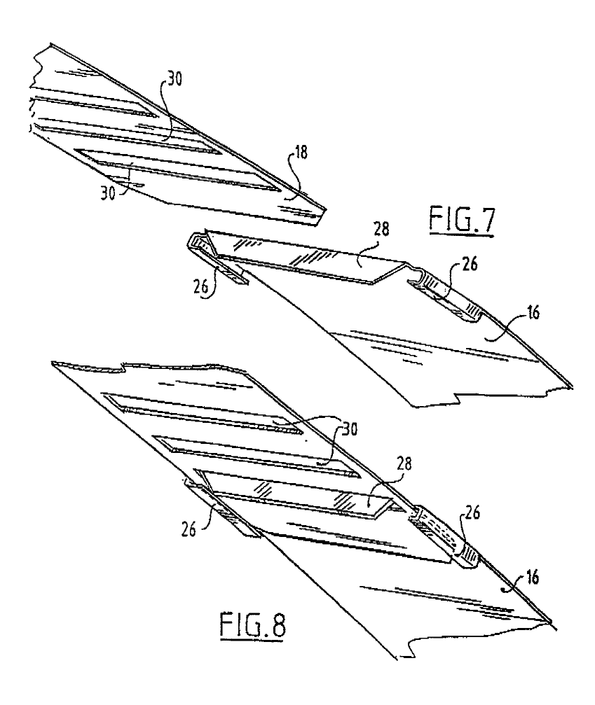

FIG. 7 shows a partially cut away perspective view of an embodiment of the releasable locking means;

FIG. 8 shows the locking means as in FIG. 7 when releasibly locked in the position;

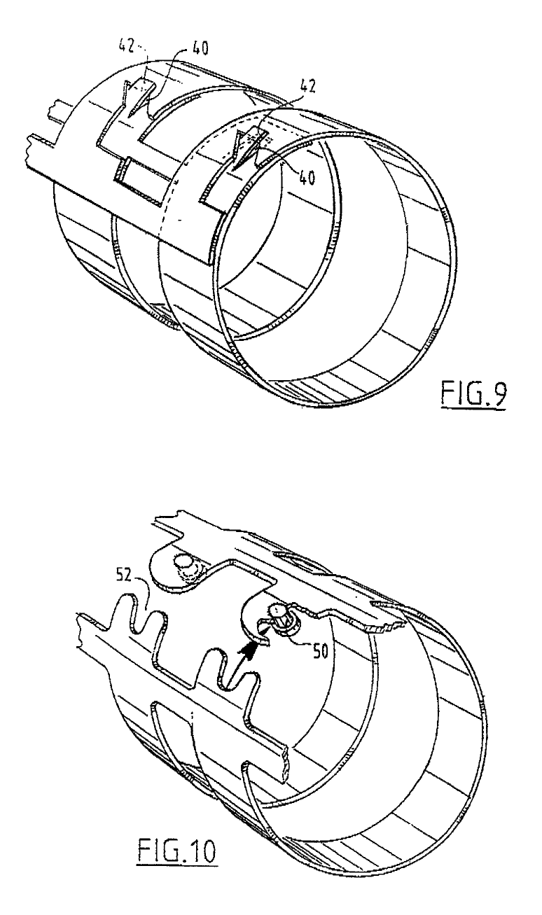

FIG. 9 shows a perspective view of a second preferred embodiment of the device according to the present invention;

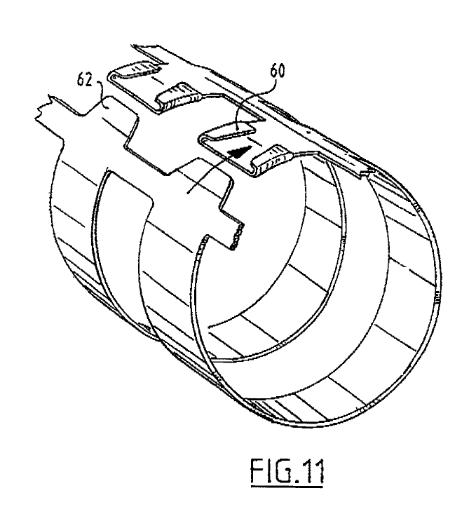

FIG. 10 shows a perspective view of a third preferred embodiment of the device according the present invention;

FIG. 11 shows a fourth preferred embodiment of the device according to the present invention;

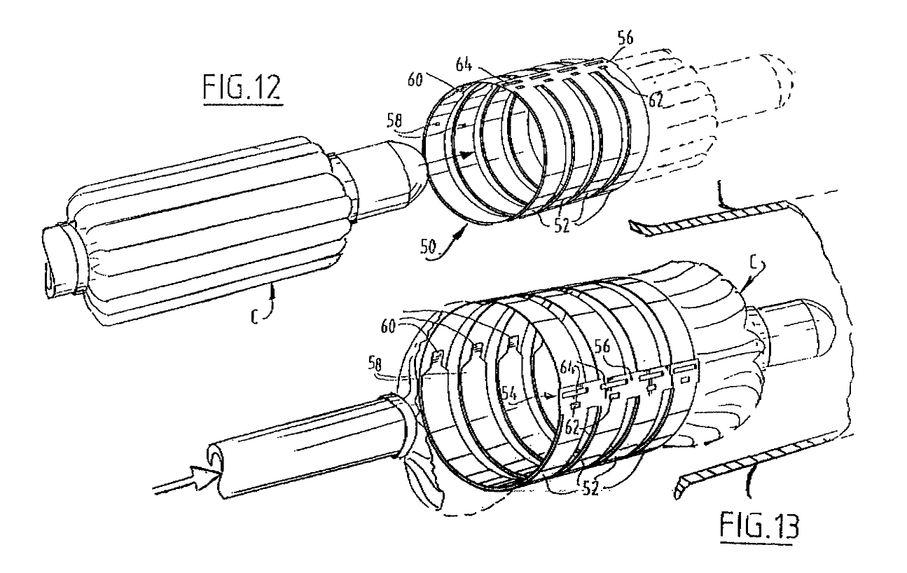

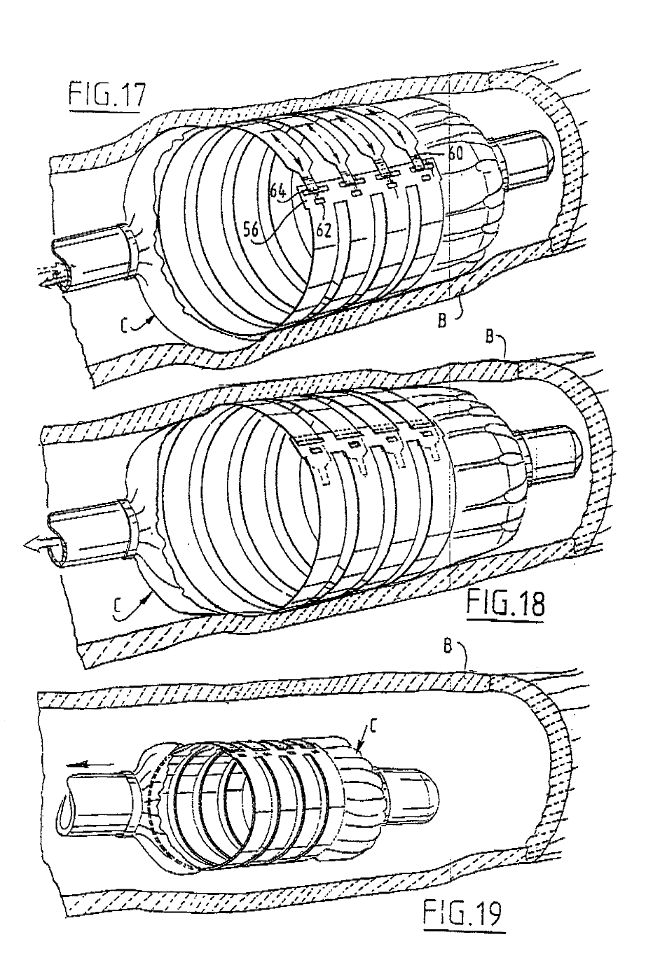

FIGS. 12-19 show perspective views of a further preferred embodiment of the present invention on insertion into, arranging in and removal from a blood vessel; and

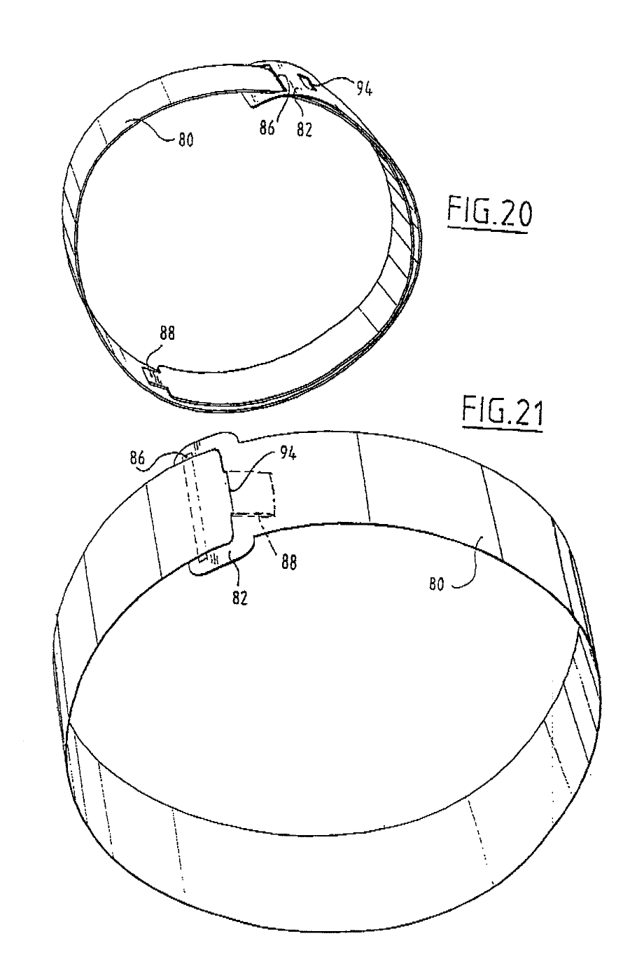

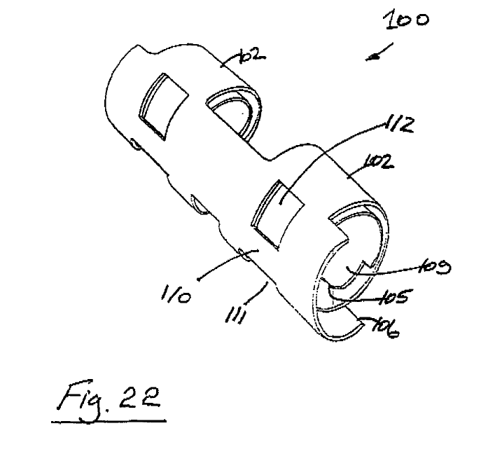

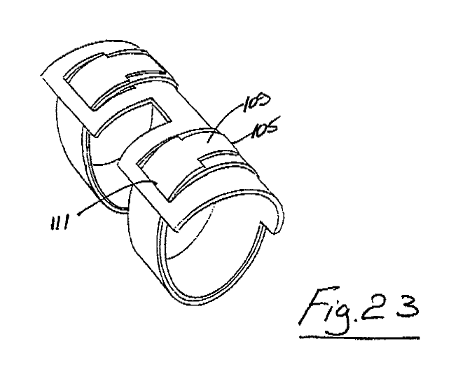

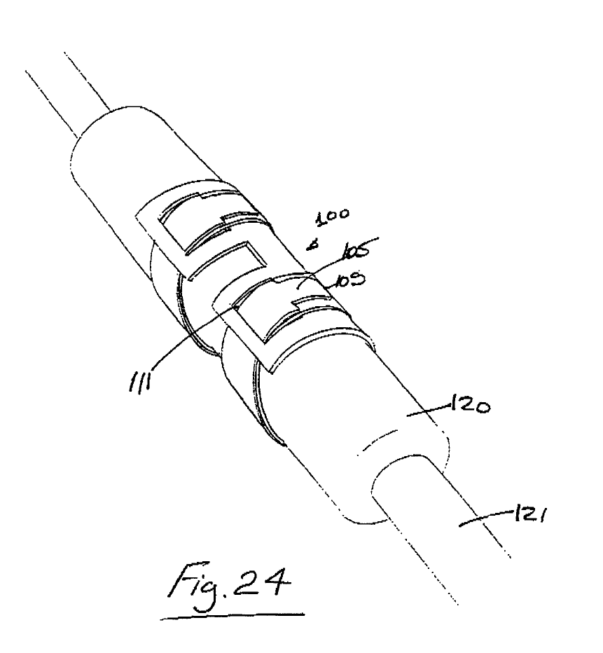

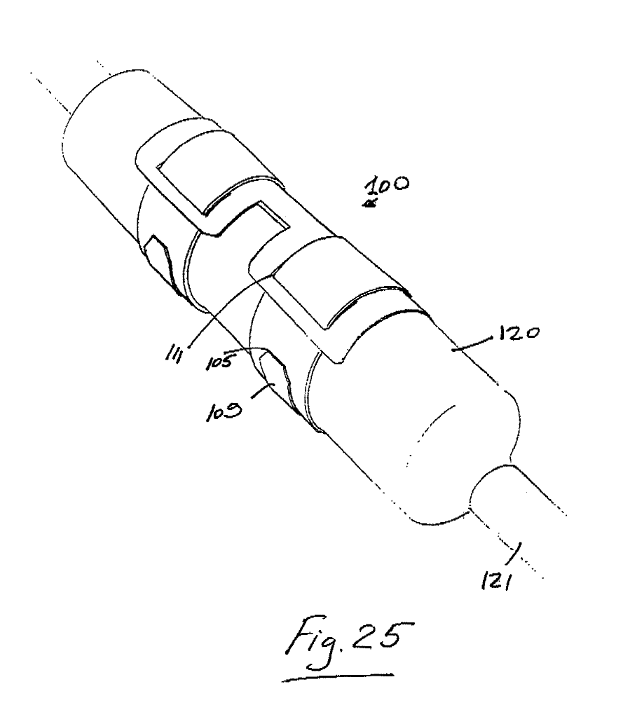

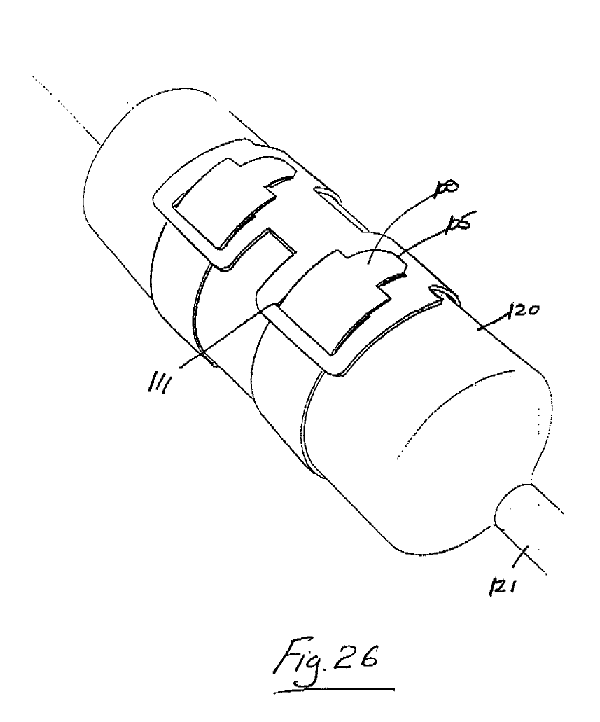

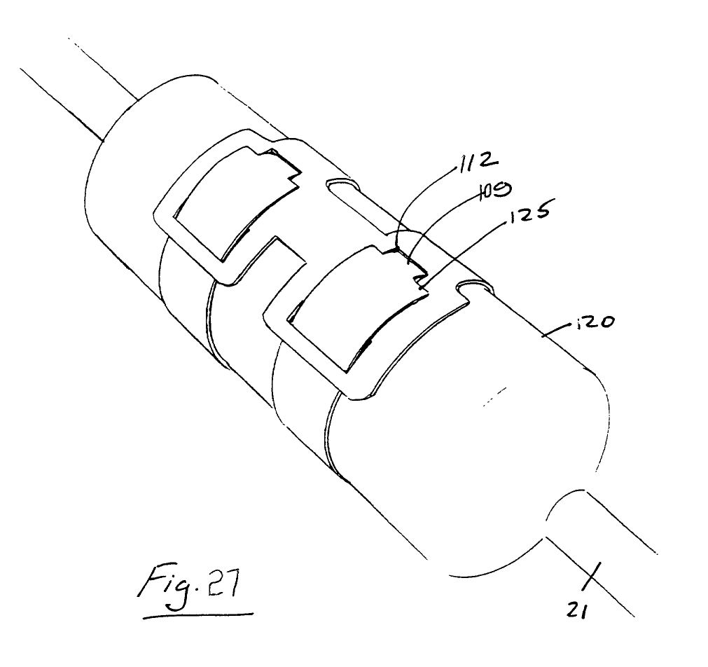

FIGS. 20 and 21 show respectively contracted and expanded perspective views of a further embodiment of the present invention,

FIG. 22 is a perspective view of a stent in a first contracted configuration;

FIG. 23 is a perspective view of the stent of FIG. 22 in a pre-tensioned configuration;

FIG. 24 is a perspective view of the pretensioned stent loaded on an introducing balloon;

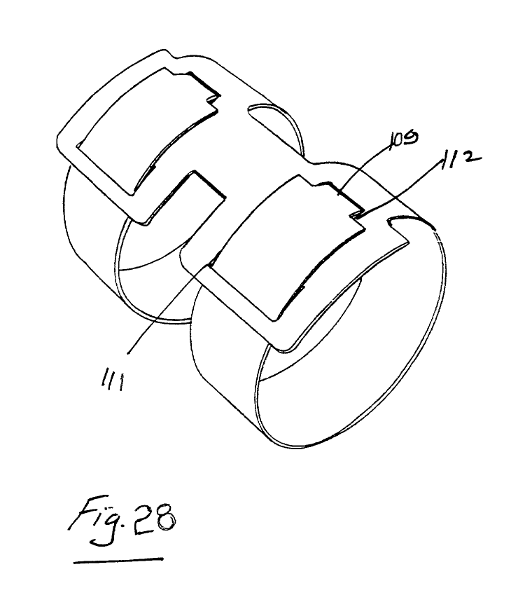

FIGS. 25 to 27 are perspective views of the stent being deployed;

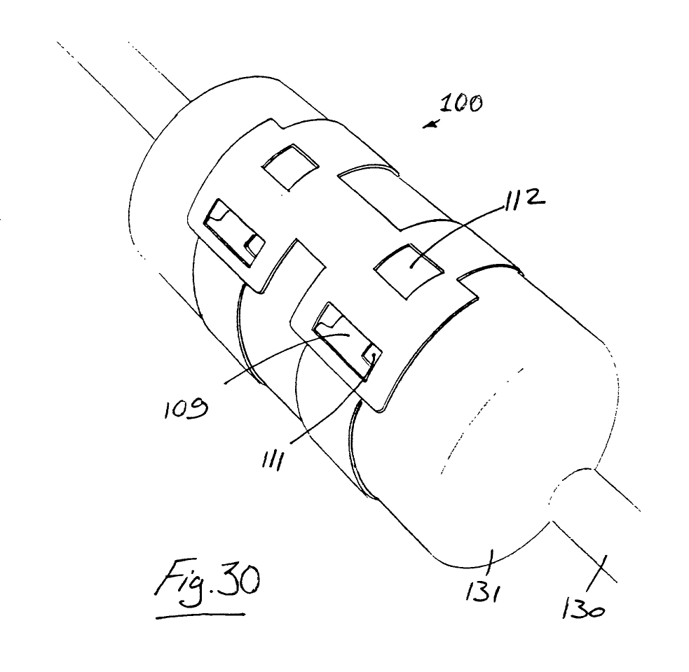

FIG. 28 is a perspective view of the deployed stent;

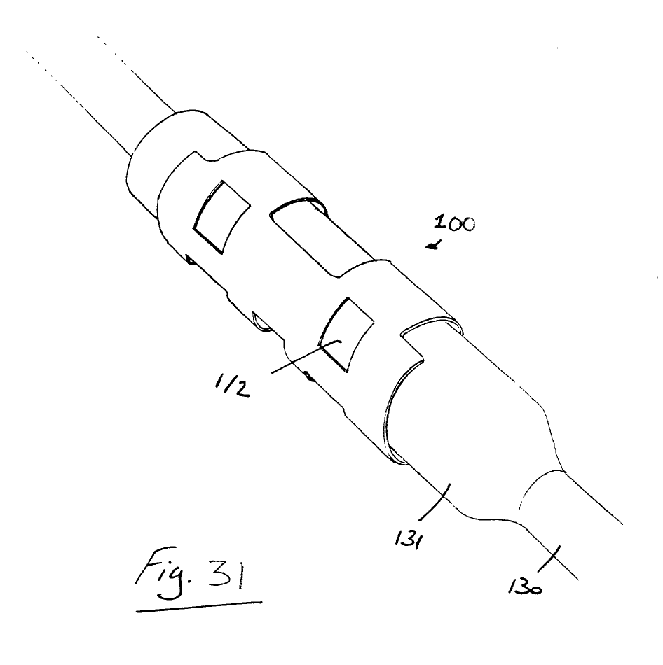

FIGS. 29 to 31 are perspective views of the stent being retrieved by a retrieval balloon; and

FIG. 32 is a perspective view of another stent according to the invention.

An assembly 1, according to the present invention, FIGS. 1, 2 and 3, has a guide wire 2 a catheter tube 4, an expandable/contractible balloon catheter 6 and a stent 10 arranged in a first contracted position to grip around the balloon catheter 6.

The stent can be pre-tensioned or made from a memory metal to assume a contracted position in its ‘resting’ state.

The balloon catheter 6 can be inflated/deflated by means of the pipes 12 in connection with the catheter tube 4.

The stent 10 consists of four ring like elements 14 each ring provided with a first terminal part 16 and a second terminal part 18 at the other end thereof (see FIGS. 4-6).

The rings 14 are formed from a single length of pre-tensioned material, for example surgical metal, whereby the terminal parts can slide over one another when transforming between the expanded/contracted arrangements. When not being used, the device is so pre-tensioned, rather like a spring, that in this ‘rest’ state it occupies a contracted state (FIG. 6).

Each ring section 14 has an outer wall 20, which outer wall 20 contacts the internal surface of the body vessel when in its expanded treating position, and an inner wall 22 which contacts to grip around the balloon catheter 6, when the device is arranged thereon, FIGS. 2, 3.

The four rings 14 are joined to provide the tubular form of the device, by means of links 24.

The first terminal part 16, of each ring 14, see particularly FIGS. 7 and 8 is provided with two laterally arranged down turned guiding lip-sections 26, and arranged there between a downturned, somewhat truncated, releasable locking edge 28.

The second terminal part 18 of the rings 14 is provided with a number of laterally arranged receiving openings 30 which are interlockable with the locking edge 28, of terminal 16.

In a second preferred embodiment of the stent (FIG. 9), the rings are joined by links arranged on a terminal part of the rings, and this terminal part is provided with a cut-away receiving section 40 which is releasably interlockable with a finger element 42 arranged on the other terminal part of the rings.

In a third preferred embodiment of the stent (FIG. 10), terminal parts of the rings are provided with depending studs 50 which are interlockable with corresponding U profiles 52 arranged on the other terminal parts of the rings.

In a fourth preferred embodiment of the stent as shown in FIG. 11, terminal ends of the rings are laterally provided with up turned cuffs 60 which co-operate with narrowing profiles 62 of the other terminal parts of the ring sections.

In use, the stent is arranged in its first contracted state, see FIGS. 2 and 4 around the deflated balloon catheter 6. In the first contracted state, the first terminal parts 16 overlap the second terminal parts 18. The assembly is then guided into position to the pre-determined treatment site within a body vessel, as shown in FIG. 1, at which treatment site the balloon catheter 6 is expanded. On inflation of the balloon catheter 6, the stent is unravelled so that the outer wall 20 thereof is pushed against the inner surface of the body vessel (see FIG. 3). The balloon catheter 6 is controllably expanded to such an extent that the terminal parts 16, respectively 18 of the rings are displaced, the rings 14 therefore expanding, so that the second terminal part 18 of the rings 14, in the first contracted state residing on the inner wall 22 of the rings 14, slips through the lips 26 of the first terminal part 16 of the rings so that the locking edge 28 falls into one of the corresponding openings 30. In this position the stent is locked in its expanded, treatment position (see FIG. 5).

The degree of expansion of the stent can be controlled by the degree of expansion of the balloon catheter. Body vessels of varying internal diameters are catered for by the presence of a plurality of receiving openings 30.

It will be clear that the embodiments as shown in FIGS. 9, 10 and 11 are expandable and lockable by the same principle.

Once the period of treatment has expired, the balloon catheter 6 can once more be inserted into the body vessel and inflated to force open the stent whereby the locking means are decoupled, i.e. whereby the locking edge 28 is forced up out of the corresponding opening 30 so that the two terminal parts 16, 18 of the rings are forced further apart until the terminal part 16 now slips beneath the terminal part 18 to re-assume a pre-tensioned second contracted state, the rings contracting to once more grip around the balloon catheter (see FIG. 6).

The balloon catheter can then be deflated whereby the two ends of the rings slide over one another until the stent re-assumes its pre-tensioned contracted position, gripping around the balloon catheter.

It will be obvious that the embodiments as shown in FIGS. 9, 10 and 11 work by exactly the same principle.

A further preferred embodiment of the device according to the present invention is shown in FIGS. 12-19.

This embodiment of the device 50 comprises four ring sections 52, each consisting of a strip of pre-tensioned material, which at a first terminal part 54 are joined together by a backbone 56 and which at a second terminal part 58 are provided with a tapered male stub 60.

Continuous with the rings 52 and arranged in the first terminal part 54 are female receiving openings 62 interlockable with the male projecting stubs 60, and guiding apertures 64 arranged between the female receiving openings 62 and a terminal edge of the first terminal part 54.

In use, the device 50 is arranged to grip around a deflated balloon catheter (see FIG. 12) in which contracted, rest position the device 50 is pre-tensioned to grip around the deflated balloon catheter C. In this first contracted position, the second terminal part 58 is not extended through the aperture 64.

Subsequently the device 50 gripping around the balloon catheter C can be inserted into a blood vessel B (see FIG. 13) and displaced therethrough until the treatment site has been reached (see FIG. 14).

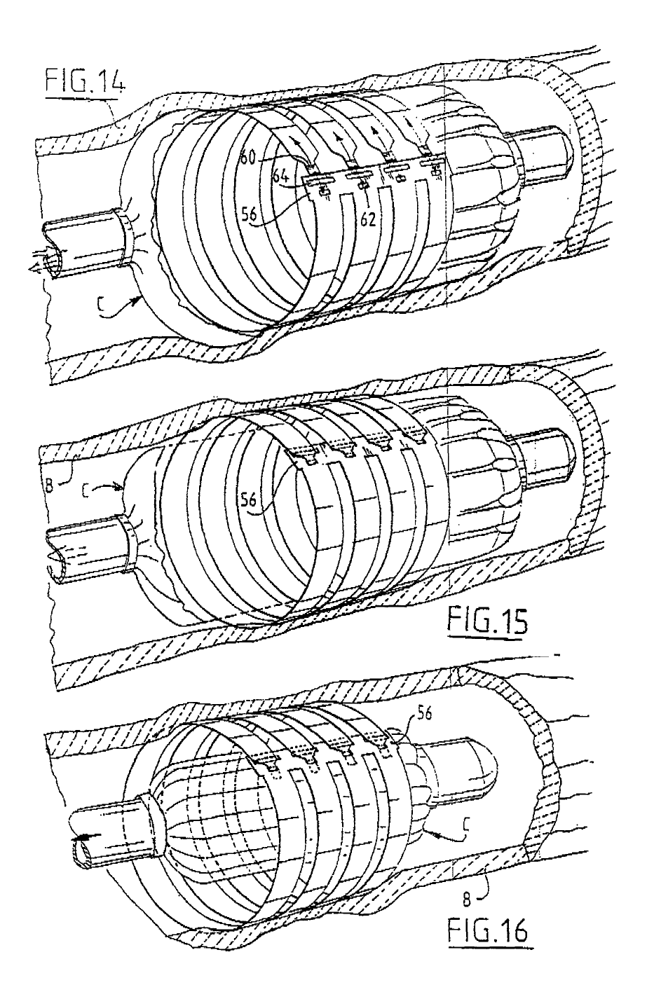

At this treatment site, the balloon catheter C can be further expanded so that the rings 52 unraffle so that the second terminal part 58 slips over the edge of the first terminal part 54 whereby the stubs 60 slip into the female receiving opening 62 (see FIG. 15) to releasably lock the rings 52 in their expanded positions (see FIG. 15). At this point the balloon catheter C can be deflated and removed from the blood vessel (see FIG. 16).

In this expanded state, the stent can be left within the blood vessel until the treatment has been completed.

Subsequently, the stent can be removed by reinserting the balloon catheter C into the blood vessel B and through the stent. Now the balloon catheter C can be once again expanded to force the male stubs 60 up and out of the female receiving openings 62 so that the male stubs 60 slide over the first terminal part 54 and into the guiding apertures 64, at which point the balloon catheter is deflated so that the second terminal part 58 is displaced through the aperture 64 on recontracting of the stent 50 to assume its second rest, contracted state (see FIG. 18 and 19). The apertures 64 ensure a good degree of safety and controllability over the recontraction of the stent. In this contracted state, the stent can then be removed from the blood vessel.

A further embodiment of the device according to the present invention is shown in FIGS. 20 and 21 and consists of a single strip of either pre-tensioned material or memory metal.

This ring strip 80 is provided at a first terminal end thereof with a female receiving opening 94 and a strip guiding aperture 86 and at a second terminal end thereof with a male projecting stub 88 which interlocks with the female receiving opening 94 in an expanded state (see FIG. 21).

When being released from this expanded state (FIG. 21), the male stub 88 slips through the receiving aperture 86 to reassume a second contracted state wherein the stent is removable (FIG. 20).

The first contracted state wherein the strip does not extend through the guiding aperture 86 is not shown.

Referring to FIGS. 22 to 32 and initially to FIG. 22 thereof an expandable stent 100 is typically manufactured from a shape memory material such as Nitinol. The stent 100 in this case comprises a series of rings 102, only two of which are illustrated. Each of the rings 102 is of generally spiral shape having a first free end 105 and a second free end 106. In the contracted configuration illustrated in FIG. 22 the first free end 105 is an inner free end and the second free end 106 is an outer free end. The first free end 105 is defined at the end of a strap portion 109 and a buckle portion 110 adjacent the second free end 106 has a first strap receiving slot 111 and a second strap receiving slot 112 which are most clearly visible in FIGS. 29 and 30. The strap portion 109 is received in the slots 111, 112 to lock the stent in various configurations as described in more detail below.

To prepare the stent 100 for use, the spiral contracted configuration of FIG. 22 is reversed by moving the inner free end 105 out over the outer free end 106 so that the first free end 105 becomes an outer free end and the second free end 106 becomes an inner free end as illustrated in FIG. 23. The stent is thereby pretensioned and is urged to return to the contracted configuration of FIG. 22. In the pretensioned configuration of FIG. 23 the strap portion 109 extends outwardly through the slot 111 in the buckle portion 110.

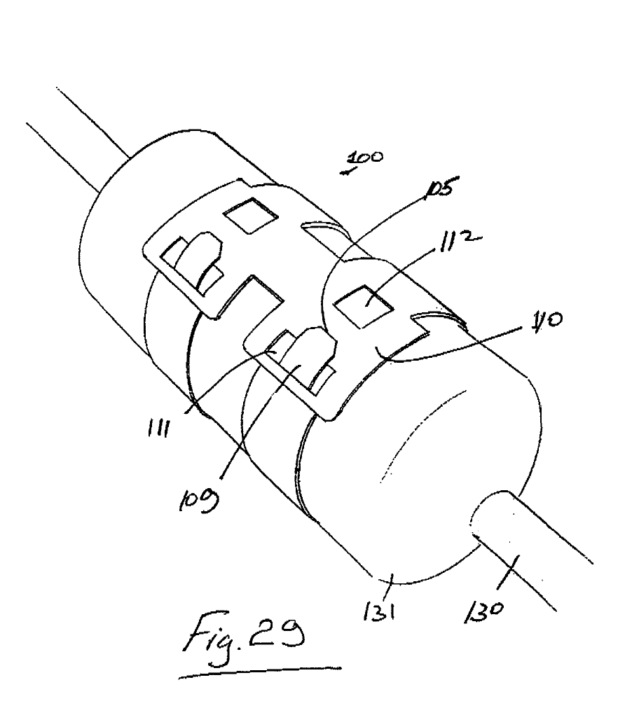

The stent in the pretensioned configuration of FIG. 23 is then loaded onto an introduction balloon 120 of an introduction catheter 121. The catheter 121 is advanced to a treatment site such as a stenosis. The introduction balloon 120 is inflated as illustrated in FIGS. 25 and 26 until the free end 105 of the strap portion 109 is aligned with the slot 112. The strap portion 109 then engages in the slot 112 and is locked in this configuration by the engagement of a shoulder 125 adjacent to the strap section 109 against the stent wall in the region of the slot 112 as illustrated in FIG. 27. By this action the stent is locked in this first expanded configuration and the balloon 120 may be deflated and the introducer catheter 121 withdrawn leaving the stent in situ as illustrated in FIG. 28.

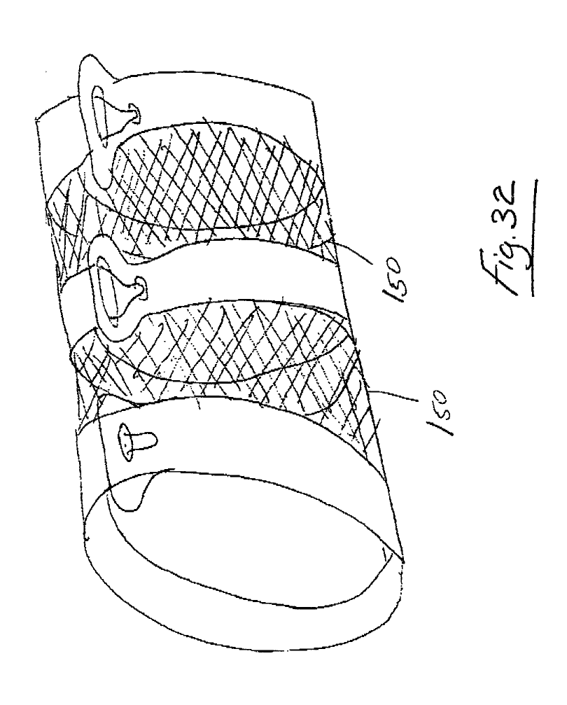

To retrieve the stent 100 a retrieval catheter 130 with a retrieval balloon 131 is advanced to the location of the stent. The retrieval balloon 131 is then inflated to expand to a diameter greater than that of the introducing balloon 120 causing the strap 109 to disengage from the slot 112 as illustrated in FIG. 29. Continued inflation of the retrieval balloon 131 pushes the slot 111 over the strap 109 until the free end 105 lies on the inside of the stent (FIG. 30). This is the manufactured configuration of the stent (FIG. 22) and on deflation of the retrieval balloon 131 the stent, by virtue of the shape memory of the material, contracts (FIG. 31). The contracted stent 100 is then removed from the vasculature carried by the retrieval balloon 131 on the retrieval catheter 130.

The stent may be used as a medicament or drug delivery device. In one embodiment of this aspect the stent is coated with a porous drug-containing coating. Alternatively or additionally a craft or tissue 150 may be used to cover at least part of the stent and/or, especially as illustrated in FIG. 32, to extend between adjacent rings. The craft or tissue may be a bioabsorbable or biodegradable material which is impregnated with a drug to be delivered to a treatment site. In some cases a layered tissue may be used. One layer may be used to carry drugs and deliver these at the site. A second layer may be used to prevent flow of drug in the opposite direction. Such a layered structure allows treatment to be delivered at the outside of the stent to the wall of the lumen, or treating the material (such as blood) that is on the inside of the stent and not the wall of the lumen. This provides an even further enhanced localised and precise treatment.

The drug delivery technique utilising the stent described is particularly advantageous as the stent and therefore the associated drug can be removed during treatment. The stent is readily delivered, used for treatment and removed.

The drugs, which may be delivered using the stent and delivery technique, may be, for example, anticoagulant or antithrombotic agents, anti-inflammatory agents and the like.

The present invention is not limited to the above described preferred embodiments; the rights of the present invention are to be determined by the following claims, in which many modification are possible.

1. Device suitable for internally supporting vessels in particular circular vessels in the non-medical and medical fields, such as transmission pipes and blood vessels, the urinary tract, the digestive tract, and airways, said device comprising:
an outer wall, and
an inner wall in association with the outer wall, whereby both the inner and outer wall are expandable and contractible between an expanded support position, in which support position the outer wall contacts an internal surface of the vessel to be supported and a contracted displacing position wherein the device is displaceable to and from a pre-desired location in the vessel, and,
releasable locking means for releasibly locking the device in the expanded support position and/or the contracted displacing position.

2. Device according to claim 1, wherein the outer and inner walls are provided in the form of one or more roughly circular elements.

3. Device according to claim 2 wherein the one or more roughly circular elements take the form of one or more rings.

4. Device according to any of the preceding claims, wherein the inner and outer walls comprise a first terminal part and a second terminal part.

5. Device according to claims 3 or 4 wherein the ring elements are interconnected by one or more linking members.

6. Device according to claim 5 wherein the releasible locking means comprise interlocking means provided on the first and/or second terminal parts.

7. Device according to claim 6 wherein the interlocking means comprise a catching element arranged on the first terminal part, said catching element being co-operable with a locking opening arranged on the second terminal part.

8. Device according to claim 7 further comprising guiding means for guiding the terminal parts over one another during expansion and/or contraction of the device.

9. Device according to claim 8 wherein the guiding means comprise one or more lip-sections in association with the first terminal part, which lip-sections cooperate with the second terminal part.

10. Device according to any of the preceding claims further provided with a tracing agent, whereby the device is traceable when arranged in position with the body by means of medical locating techniques such as magnetic resonance imaging.

11. Device according to any of the claims 1-10 further provided with a radio-active material in order to provide localized radiation therapy.

12. Device according to any of the preceding claims further comprising a medicament in order to locally treat a medical disorder within the body.

13. Device according to any of the preceding claims, the device being pre-tensioned to assume in its resting state, either the contracted position or the expanded position, preferably the contracted position.

14. Device according to any of the preceding claims, said device comprising a memory metal which assumes the contracted and/or expanded portion when exposed to certain conditions.

15. Device according to any of the preceding claims wherein when occupying a removal displacing position, the first terminal part is overlapped by the second terminal part and when occupying an insertion displacing position, the second terminal position is overlapped by the first terminal position.

16. Device according to any of the preceding claims wherein the locking means comprise a male projection arranged on the second terminal part, interlockable with a female receiver arranged on the first terminal part.

17. Device according to claim 16, further comprising an aperture on the first terminal part, through which the second terminal part is transposeable.

18. Assembly for treating body vessel disorders, said assembly comprising a device according to any of the preceding claims, and,
introducing and/or removing means for introducing and or removing the device to and/or from the desired location within a vessel.

19. Assembly according to claim 18 wherein the introducing and or removal means comprise and expandable/deflateable balloon catheter.

20. Process for arranging a device according to any of the claims 1-17 within a body vessel, comprising the steps of,
arranging the device in its contracted form around a balloon catheter, so that the device grips onto the balloon catheter,
bringing the balloon catheter plus contracted device to a predetermined position with a body vessel. expanding the balloon catheter whereby the device is also expanded, to such an extent that the releasably locking means are locked in position, whereby in this expanded use position the balloon catheter may optionally be deflated and removed.

21. Process according to claim 20 further comprising the steps of
reintroducing a balloon catheter into the vessel,
expanding the balloon catheter against the inner wall of the device to such an extent that the device is further expanded in order to release the releasable locking means,
followed by deflating the balloon catheter whereby the device re-assumes its contracted position to grip around the balloon catheter,
whereafter the balloon catheter and device may be removed from the body vessel.

22. Method for treating vessels, for example transmission pipes or body vessels, utilizing a device according to any of the claims 1-17 and or the assembly according to the claims 18 or 19.

23. Use of a device according to any of the claims 1-17 and/or the assembly according to claims 18 or 19 for treating vessels of the digestive tract.

24. Use of the device according to any of the claims 1 to 17 and/or the assembly according to claims 18 or 19 for treating vessels of the urinary tract.

25. Use of the device according to any of the claims 1 to 17 and/or the assembly according to claims 18 or 19 for treating the vessels of the airways, such as the trachaea and bronchii, in particular in brachy therapy.

26. Use of the device according to any of the claims 1 to 17 and/or the assembly according to claims 18 or 19 for treating blood vessels such as arteries and/or veins.

27. Use of the device according to any of the claims 1 to 17 and/or the assembly according to claims 18 or 19 for locally radioactively treating a body vessel.

28. Use of the device according to any of the claims 1-17 and/or the assembly according to claim 15 or 16 for non-medical application, in particular for internally supporting transmission pipes and the like.

29. A method for preparing an expandable stent comprising the steps of:
forming a generally spiral shaped element having a first free end and a second free end, the spiral element having a contracted configuration in which the first free end is an inner free end and the second free end is an outer free end; and
reversing the spiral so that the first free end becomes an outer free end and the stent is pretensioned.

30. A method for operating an expandable stent in the form of a generally spiral-shaped element the method comprising the steps of:
delivering the stent in the pretensioned configuration to a desired site;
deploying the stent by expanding the stent to a first expanded configuration; and
retrieving the stent by expanding the stent further to a second expanded configuration.

31. A method for manipulating a stent having a pretensioned contracted configuration, a first expanded configuration and a second expanded configuration, the method comprising the steps of:
delivering the stent in a pretensioned contracted form to a desired site;
deploying the stent by expanding the stent to the first expanded configuration; and
subsequently retrieving the stent by further expanding the stent to the second expanded configuration whereby the stent collapses.

32. A method as claimed in claim 31 wherein the pre-tensioned contracted stent is mounted on an introduction balloon catheter and the method includes the steps of advancing the balloon catheter to a desired site and inflating the introduction balloon to deploy the stent.

33. A method as claimed in claim 31 or 32 wherein the stent is retrieved by advancing a retrieval balloon catheter to the stent and inflating the retrieval balloon to expand the deployed stent to the second expanded configuration whereby the stent collapses.

34. An expandable stent comprising a generally spiral shaped element having a pretensioned configuration for delivery to a desired site, a first expanded configuration for deployment of the stent, and a second expanded configuration for retrieval of the stent, the stent having a medicament delivery system for delivery of a medicament at a target site.

35. A stent as claimed in claim 34 wherein the medicament system comprises a coating containing a medicament.

36. A stent as claimed in claim 34 or 35 wherein the medicament delivery system comprises a craft or tissue containing a medicament.

37. A stent as claimed in claim 36 wherein the tissue or craft is of layered construction.

38. A stent as claimed in claim 37 wherein the tissue or craft comprises a first layer for drug delivery in one direction and a second layer to prevent drug delivery in an opposite direction.

39. A stent as claimed in any of claims 36 to 38 wherein the stent comprises a number of rings extending from a spine and the craft or tissue extends at least some of the adjacent rings.

40. A method for delivery of a drug medicament to a target site comprising
providing a stent with a medicament delivery system;
delivering the stent to a desired site;
deploying the stent to deliver the medicament; and
retrieving the stent.

41. A method as claimed in claim 40 wherein the stent is a stent as claimed in any of claims 34 to 39.
